(19) United States
(12) Patent Application Publication
(10) Pub. No.: US 2020/0253764 A1
(43) Pub. Date: Aug. 13, 2020

(54) METHOD AND SYSTEM FOR DEPLOYING A STENT ALONG AN ANGLED BRANCH VESSEL OF A MAIN VESSEL JUNCTURE

(71) Applicant: David C. LEW, Groveland, FL (US)

(72) Inventor: David C. LEW, Groveland, FL (US)

(21) Appl. No.: 16/788,688

(22) Filed: Feb. 12, 2020

Related U.S. Application Data
(60) Provisional application No. 62/804,472, filed on Feb. 12, 2019.

(51) Int. Cl. A61F 2/958; A61F 2/954; A61F 2/82

(52) U.S. Cl. CPC A61F 2/958; A61F 2002/30948; A61F 2/82; A61F 2/954

(57) ABSTRACT

A method is provided for deploying a stent along an angled branch vessel of a vessel juncture. The method includes determining a first non-orthogonal angle between the angled branch vessel and a main vessel. The method includes providing a stent with an end face forming a second non-orthogonal angle with a longitudinal axis, where the second non-orthogonal angle is based on the first non-orthogonal angle. The method includes moving the stent along a guidewire in the angled branch vessel to a constriction in the angled branch vessel. The method also includes inflating a balloon to move the stent to an extended position against the wall of the angled branch vessel, where the end face does not extend into the main vessel. The method also includes deflating the balloon and retracting the balloon from the angled branch vessel and removing the guidewire from the angled branch vessel. A stent and system are also provided associated with the method.

### CROSS REFERENCE TO RELATED APPLICATIONS

This application claims benefit of Provisional Application No. 62/804,472, filed Feb. 12, 2019, the entire contents of which is hereby incorporated by reference as if fully set forth herein, under 35 U.S.C. § 119(e).

### BACKGROUND

In medicine, a stent 108 (FIG. 1A) is a metal or plastic tube inserted into the lumen of an anatomic vessel or duct to keep the passageway open, and stenting is the placement of a stent. There is a wide variety of stents used for different purposes, from expandable coronary, vascular and biliary stents, to simple plastic stents used to allow the flow of urine between kidney and bladder. “Stent” is also used as a verb to describe the placement of such a device, particularly when a disease such as atherosclerosis has pathologically narrowed a structure such as an artery.

As illustrated in FIG. 1A, conventional systems 100 feature a guidewire 102 that is positioned in the vessel and the stent 108 (along with a deployment mechanism, e.g. balloon 104 with a balloon tip 105 that engages the guidewire 102) is moved along the guidewire to a deployment site in the vessel. The balloon 104 is inflated at the deployment site which causes the stent 108 to move from a compressed stent 108 with a first diameter to a deployed stent 106 with a second diameter greater than the first diameter. The inflated balloon 104′ has a larger diameter than the balloon 104 prior to inflation.

As appreciated by one skilled in the art, the deployed stent 106 is permanently secured against the vessel wall. In one example, plaque is along the vessel wall at the deployment site that affects a flow of fluid within the vessel. In this example, the balloon 104 is used to deploy the stent 106 against the plaque and the vessel wall, to open up the vessel so that the flow of fluid is not affected by the plaque. In another example, a diameter of the vessel narrows at the deployment site that affects a flow of fluid. In this example, deployment of the stent 106 increases the diameter of the vessel so that the flow of fluid is not affected by this vessel narrowing.

### SUMMARY

The current inventor has recognized that deployment of the conventional stent 106 in vessels has notable disadvantages, particularly in an angled branch vessel of a main vessel juncture, e.g. a bifurcation. FIG. 1B is an image that illustrates an example of the conventional stent 108 of FIG. 1A being moved along a main vessel 112 and into an angled branch vessel 114 of a vessel juncture 115, e.g. bifurcation. The vessel juncture 115 is defined by a non-orthogonal angle 110 between the angled branch vessel 114 and the main vessel 112.

In one example, the angled branch vessel 114 features a constriction opposite from a fork or bifurcation 118 of the angled juncture 115. In one embodiment, the constriction is plaque 116 along a wall 120 of the angled branch vessel 114 opposite from a fork or bifurcation 118 of the angled juncture 115. In an example embodiment, the plaque 116 is within a range from about 2 millimeters (mm) to about 3 mm and/or within a range from about 1 mm to about 5 mm of the main vessel 112. In another example, the constriction is based on the wall 120 of the angled branch vessel 114 narrowing in the area of the bifurcation 118 of the vessel juncture 115. The plaque 116 affects a flow of fluid (e.g. blood) through the angled branch vessel 114 and into or out of the vessel juncture 115. The compressed stent 108 is moved along the guidewire 102 to the deployment site (FIG. 1C) such that the end 122 of the compressed stent 108 is at or beyond a distal end of the plaque 116. The balloon 104a is then inflated (FIG. 1D) to deploy the stent 106 against the interior wall 120 of the angled branch vessel 114 to open up the angled branch vessel 114 and/or prevent the plaque 116 from affecting a fluid flow through the angled branch vessel 114 and/or from protruding into the angled branch vessel 114. As depicted in FIG. 1E, the balloon 104a is subsequently deflated and removed from the angled branch vessel 114 along the guidewire 102 through the main vessel 112.

The inventor of the present invention noticed that although the conventional deployed stent 106 advantageously opens up the angled branch vessel 114, the conventional deployed stent 106 introduces several drawbacks. For example, a flow of fluid (e.g. blood flow 125) in the main vessel 112 is affected by the end 122 (e.g. corner 123) of the stent 106 that extends into the main vessel 112. The inventor of the present invention recognized that conventional techniques employ a secondary balloon 104b that is moved along a secondary guidewire 102b (FIG. 1F) placed in the main vessel 112. The secondary balloon 104b is moved along the secondary guidewire 102b to the area where the stent 106 extends into the main vessel 112 (FIG. 1G). The secondary balloon 102b is then inflated in an attempt to move the end 122 (e.g. corner 123) of the deployed stent 106 out of the main vessel 112, so that the flow of fluid (e.g. blood flow 125) in the main vessel 112 is not affected by the deployed stent 106. However, the inventor of the present invention noticed that this introduces additional drawbacks, notably that the deployed stent 106 is compressed into a compressed end 107 which now extends into the angled branch vessel 114 at the juncture 115 and affects a flow of fluid along the angled branch vessel 114. Thus, as shown in FIG. 1G, conventional systems involve moving the balloon 104a from the main vessel 112 back along the guidewire 102a second time to the area of the compressed end 107 to inflate the balloon 104a a second time in an attempt to move the compressed end 107 out of the angled branch vessel 114.

The inventor of the present invention recognized that the conventional method to deploy the stent 106 at the vessel juncture 115 is deficient, since it involves several steps and numerous components, including the use of multiple balloons 104a, 104b, positioned in multiple vessels 112, 114, as well as multiple balloon inflations. Additionally, the inventor of the present invention noted that even with all of these numerous steps and numerous components, the conventional method results in limited success since fluid flow is still affected in the angled branch vessel 114 (e.g. by the compressed end 107) and/or the main vessel 112 (e.g. by the end 122 of the deployed stent 106). The inventor of the present invention developed an improved stent to be deployed in the angled branch vessel 114 at the vessel juncture 115 (e.g. to resolve plaque 116 along the interior wall and/or origin of the angled branch vessel 114 and/or to eliminate the reduced inner diameter of the wall 120 of the angled branch vessel 114 at the vessel juncture 115), where the improved stent can be deployed with fewer steps, fewer components and is far more effective at improving fluid flow through the main vessel 112 and the angled branch vessel 114 of the vessel juncture 115.

In a first set of embodiments, a method of deploying a stent along an angled branch vessel of a vessel juncture. The method includes determining a first non-orthogonal angle between the angled branch vessel and a main vessel of the vessel juncture, where the angled branched vessel has a constriction adjacent to the vessel juncture. The method also includes providing a stent with an end face forming a second non-orthogonal angle with a longitudinal axis of the stent, where the second non-orthogonal angle is based on the first non-orthogonal angle of the vessel juncture. The method further includes moving the stent along a guidewire in the angled branch vessel to the constriction in the angled branch vessel. The method further includes inflating a balloon positioned between the stent and the guidewire to move the stent from a compressed position to an expanded position against the wall of the angled branch vessel, where the end face does not extend into the main vessel of the vessel juncture. The method further includes deflating the balloon and retracting the balloon from the angled branch vessel along the guidewire. The method further includes removing the guidewire from the angled branch vessel.

In a second set of embodiments, a stent is provided for deployment along an angled branch vessel of a vessel juncture. The vessel juncture includes the angled branch vessel and a main vessel with a first non-orthogonal angle between the angled branch vessel and the main vessel. The stent includes a proximal end face and a distal end face. The stent also includes a central portion between the proximal end face and the distal end face, where the central portion defines a longitudinal axis. The proximal end face forms a second non-orthogonal angle relative to the longitudinal axis that is based on the first non-orthogonal angle.

In other embodiments, a system or apparatus or computer-readable medium is configured to perform one or more steps of the above method.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is an image that illustrates an example of a conventional stent being deployed by a balloon positioned along a guidewire;

FIG. 1B is an image that illustrates an example of the conventional stent of FIG. 1A being moved along a main vessel of a main vessel juncture;

FIG. 1C is an image that illustrates an example of the conventional stent of FIG. 1A being moved to a deployment site at an origin and along an angled branch vessel of a main vessel juncture;

FIG. 1D is an image that illustrates an example of the conventional stent being deployed at the deployment site of FIG. 1C;

FIG. 1E is an image that illustrates an example of the deployed conventional stent of FIG. 1D partially obstructing blood flow in a main vessel of the vessel juncture;

FIGS. 1F-1G are images that illustrate an example of a secondary balloon being moved along a secondary guidewire in the main vessel of the juncture to the deployment site;

FIG. 2A is an image that illustrates an example of an assembly including a balloon, a guidewire and a stent, according to an embodiment;

FIG. 2B is an image that illustrates an example of the assembly of FIG. 2A positioned in a main vessel of a main vessel juncture, according to an embodiment;

FIG. 2C is an image that illustrates an example of the stent of the assembly of FIG. 2A moved to a deployment site along the angled branch vessel of the main vessel juncture, according to an embodiment;

FIG. 2D is an image that illustrates an example of the balloon of the assembly of FIG. 2A being inflated to deploy the stent along the wall of the angled branch vessel, according to an embodiment;

FIG. 2E is an image that illustrates an example of the balloon of the assembly of FIG. 2A being deflated after deployment of the stent along the wall of the angled branch vessel, according to an embodiment;

FIG. 2F is an image that illustrates an example of the deflated balloon of FIG. 2E being removed from the angled branch vessel along the guidewire, according to an embodiment;

FIG. 2G is an image that illustrates an example of the deployed stent of FIG. 2D that does not obstruct a blood flow within the main vessel of the main vessel juncture, according to an embodiment;

FIG. 3 is a block diagram that illustrates an example of a system for forming the stent of the assembly of FIG. 2A, according to an embodiment; and

FIG. 4 is a flowchart illustrates an example of a method for deploying a stent along an angled branch vessel of a main vessel juncture, according to an embodiment.

### DETAILED DESCRIPTION

A method and apparatus and system are described for forming a stent for deployment in an angled branch vessel of a vessel juncture (e.g. bifurcation) and for deploying a stent in an angled branch vessel of a main vessel juncture. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

For purposes of this description, “vessel juncture” means a junction of an angled branch vessel and a main vessel at a non-orthogonal angle. The vessel juncture is in a human subject or non-human subject (e.g. animal). In some embodiments, the vessel juncture includes one or more angled branch vessels joining the main vessel at the junction. In an example embodiment, a “bifurcation” is a vessel juncture where one angled branch vessel joins the main vessel at the junction. In an embodiment, “vessel” means a blood vessel that is part of the circulatory system and microcirculation that transports blood throughout the human body (or non-human body, e.g. animal body). “Angled branch vessel” means a vessel that has one end that terminates at the vessel juncture and “main vessel” means a vessel that extends continuously through the vessel juncture. In another embodiment, “angled branch vessel” has an inner diameter that is smaller than an inner diameter of the “main vessel”. “Non-orthogonal angle” means an angle other than 90 degrees and/or an angle greater than 0 degrees and less than 90 degrees and/or an angle in a range between about 1 degree and about 89 degrees and/or an angle in a range between about 10 degrees and about 80 degrees and/or an angle in a range between about 20 degrees and about 70 degrees and/or an angle in a range between about 30 degrees and about 60 degrees and/or one of a plurality of discrete angles (e.g. 30 degrees, 45 degrees, 60 degrees) that are spaced apart by an interval (e.g. 15 degrees) and within one or more of the above ranges.

Some embodiments of the invention are described below in the context of deployment of stents in an angled branch vessel of a vessel juncture. In other embodiments, the invention is described in the context of deployment of a pulmonary stent. In other embodiments, the invention is described below in the context of deploying a stent at an origin or ostium of an angled side branch originating from a main parent artery.

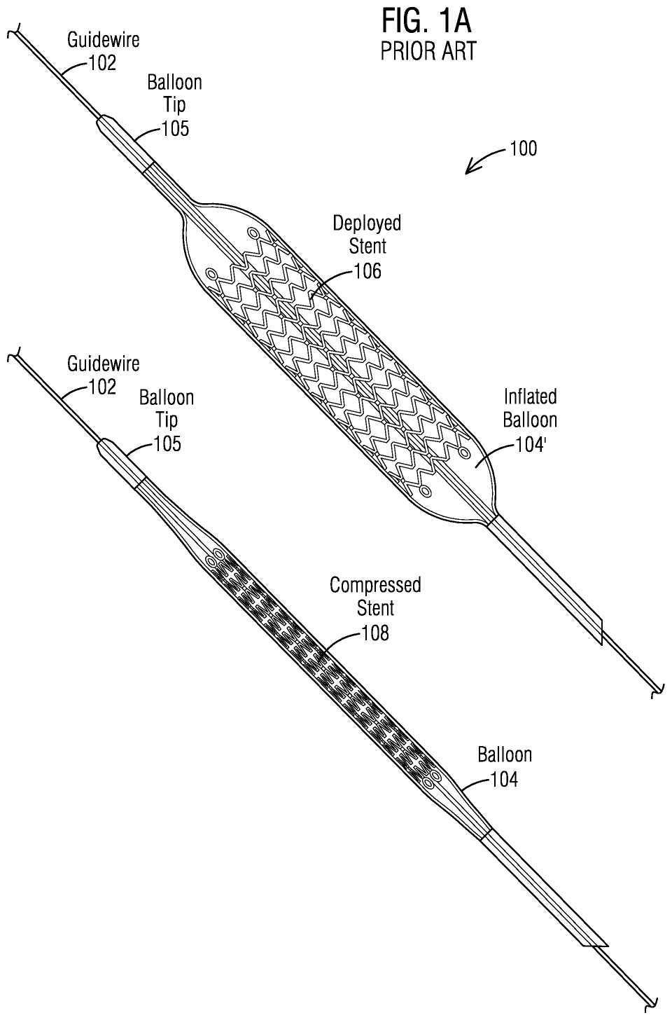

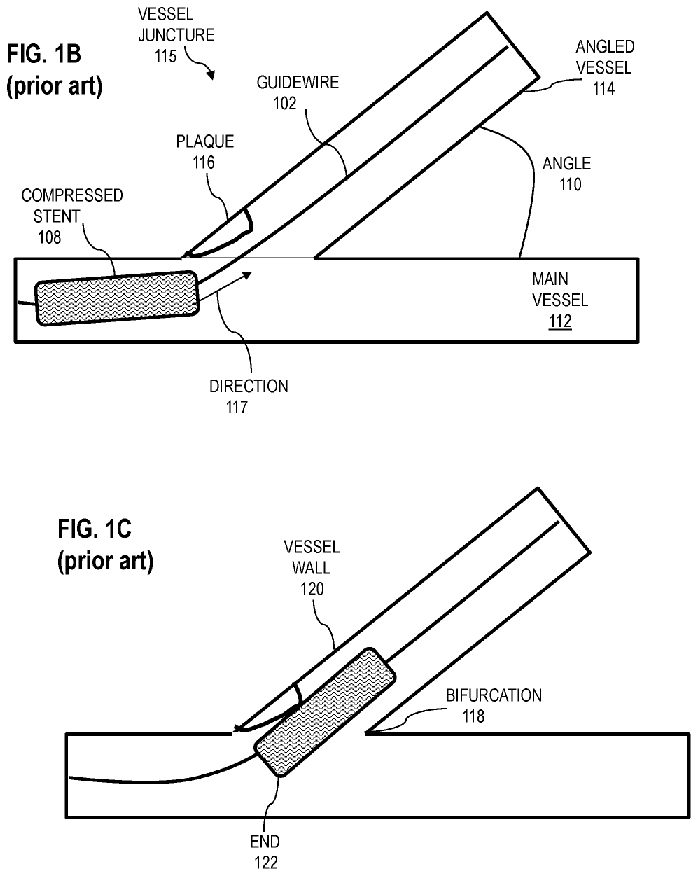

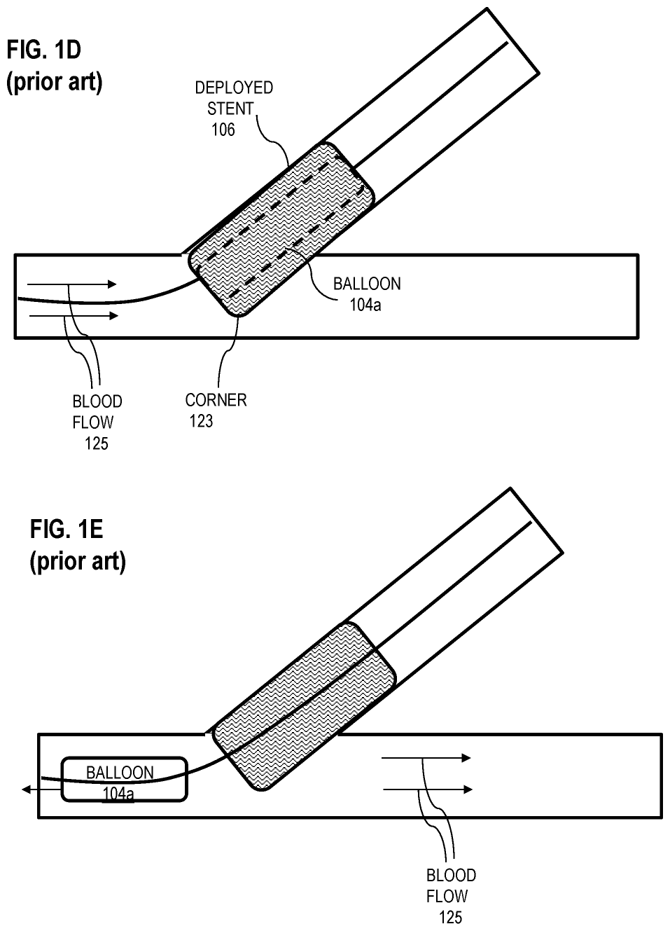

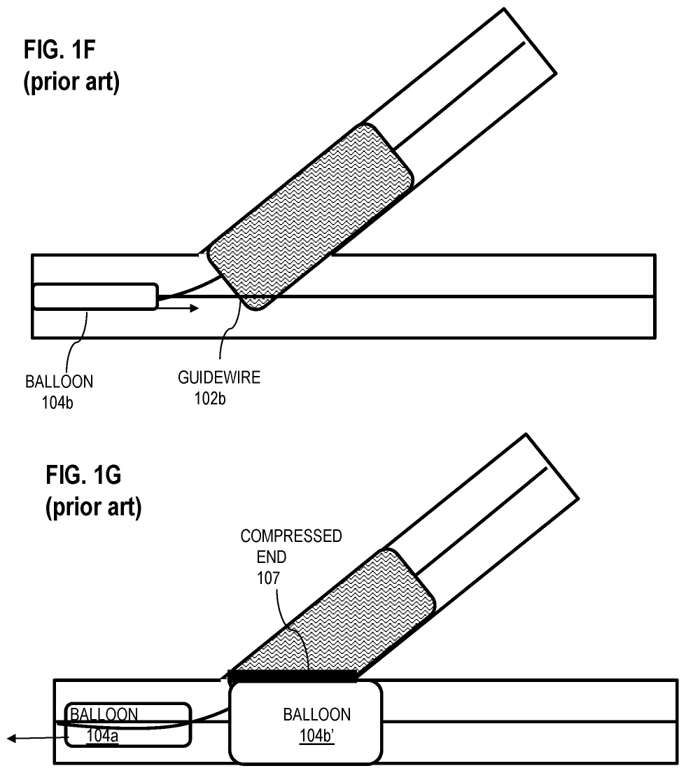

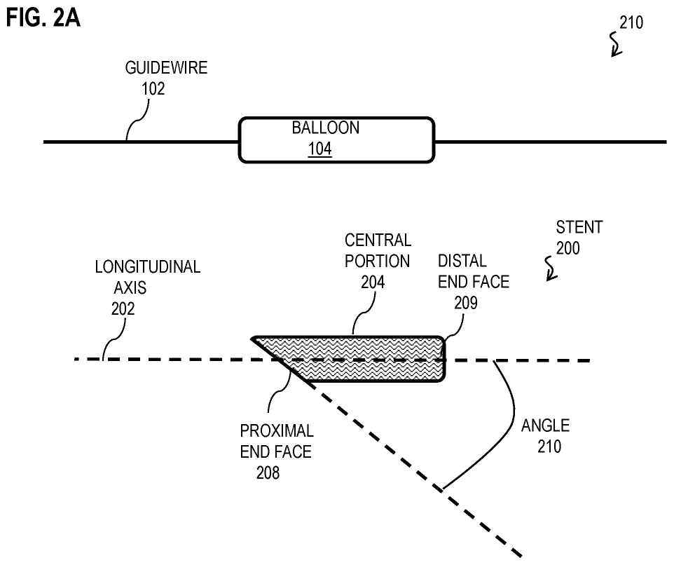

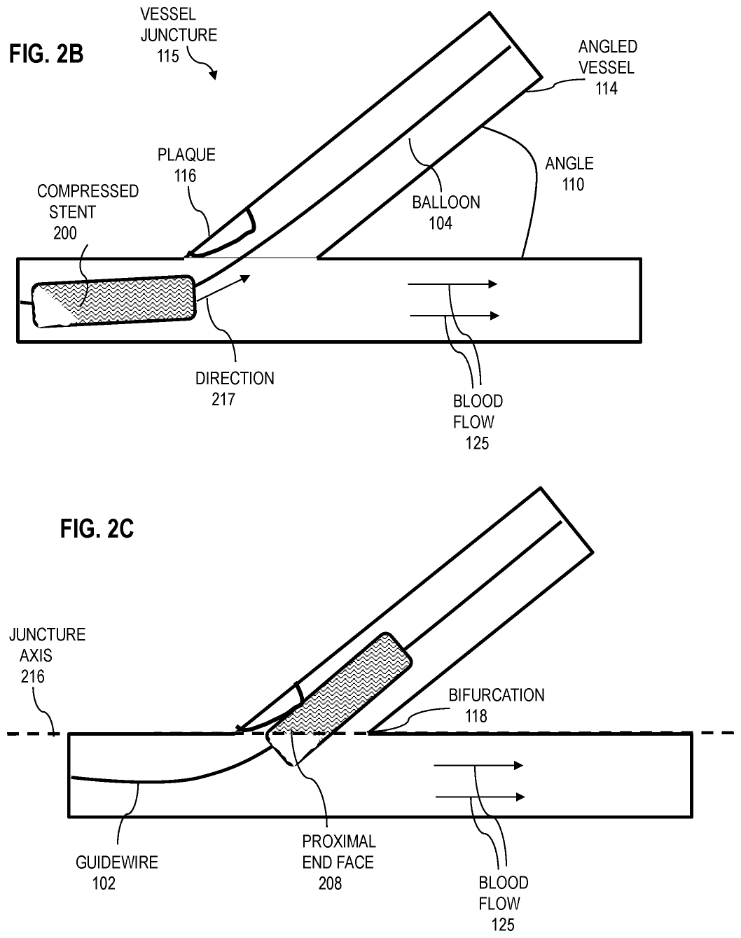

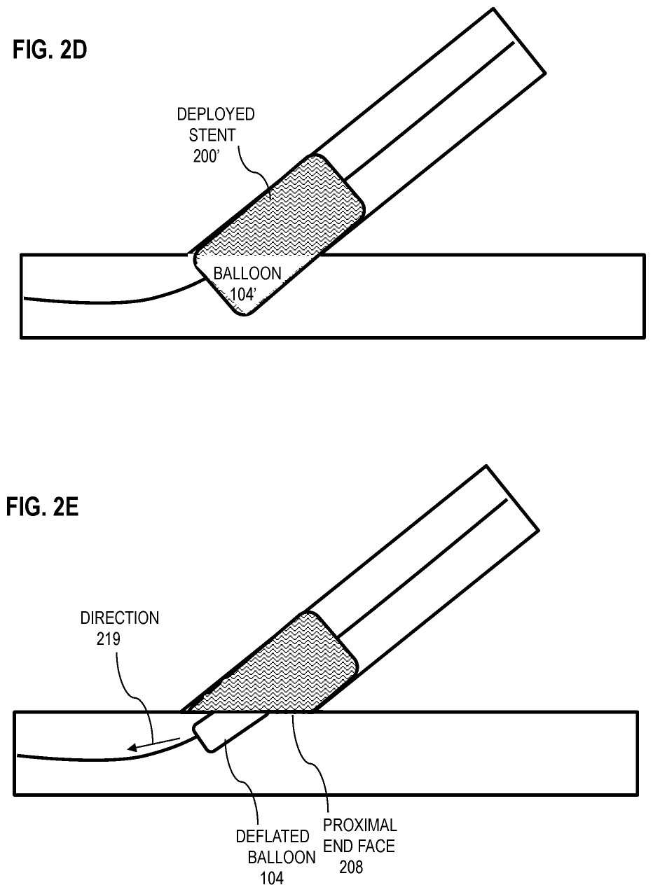

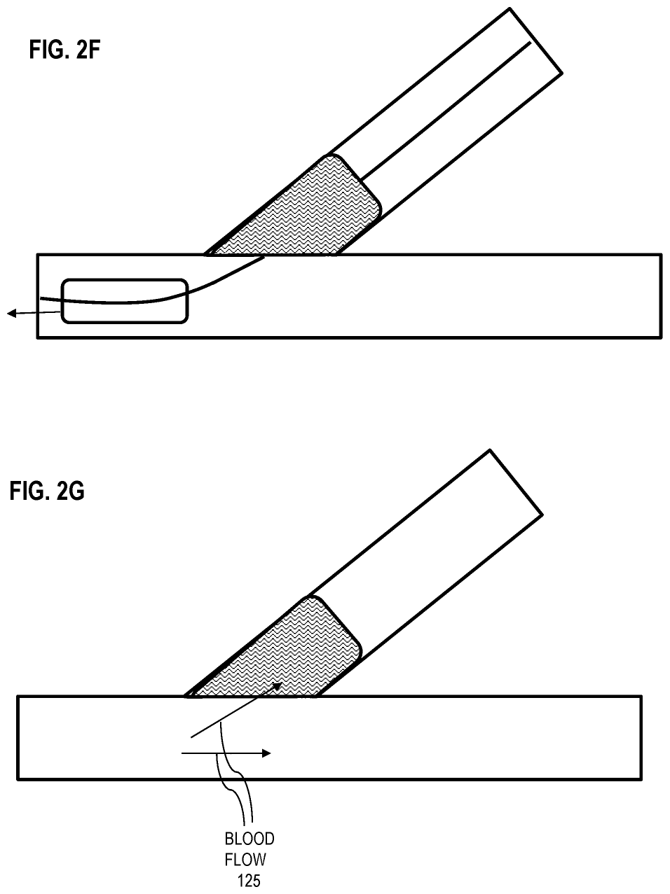

FIG. 2A is an image that illustrates an example of an assembly 210 including a deployment mechanism (e.g. balloon 104), a guidewire 102 and a stent 200, according to an embodiment. In an embodiment, the balloon 104 and guidewire 102 are similar to those used in the conventional deployment method of FIGS. 1B-1G.

In an embodiment, the stent 200 includes a distal end face 209, a proximal end face 208 and a central portion 204 between the end faces 208, 209 that defines a longitudinal axis 202 of the stent 200. In one embodiment, the stent 200 is inserted into the angled branch vessel 114 so that the proximal end face 208 is more proximate to the vessel juncture 115 than the distal end face 209. In an embodiment, the distal end face 209 is shaped similar to the distal end face in the conventional stent 100 (FIG. 1A), e.g. oriented at about 90 degrees relative to the longitudinal axis 202 and the central portion 204 is cylindrical in shape. In another embodiment, the proximal end face 208 is oriented at a non-orthogonal angle 210 relative to the longitudinal axis 202. In some embodiments, the non-orthogonal angle 210 is based on the non-orthogonal angle 110 of the vessel juncture 115. In an embodiment, the stent 200 is made from one or more of metal alloy, bio-absorbing and/or bio-degradable materials (e.g. magnesium, poly-lactic acid, etc.), plastic material and/or mesh material. In still other embodiments, the distal end face 209 forms a non-orthogonal angle with the longitudinal axis 202 (e.g. a non-orthogonal angle different than the non-orthogonal angle 210) so that the stent 200 can accommodate more than one vessel juncture 115.

FIG. 2B is an image that illustrates an example of the assembly 210 of FIG. 2A positioned in an angled branch vessel 114 of a vessel juncture 115, according to an embodiment. In an embodiment, the vessel juncture 115 is a bifurcation since only one angled branch vessel 114 is connected to the main vessel 112 at the juncture 115. However, in other embodiments, more than one angled branch vessel 114 can be connected to the main vessel 112 at the juncture 115. In these embodiments, the stent 200 can be deployed in one or more of these angled branch vessels 114, depending on whether plaque 116 is present in one or more of these angled branch vessels 114 at the juncture 115. In one example embodiment, the angled branch vessel 114 is an angled side branch of a main parent artery and the vessel juncture 115 is an origin or ostium of the angled side branch of the main parent artery.

The compressed stent 200 is positioned over the balloon 104 and guidewire 102 and inserted into the main vessel 112 over the guidewire 102, after which the stent 200 is moved in the direction 217 and into the angled branch vessel 114. In an embodiment, the stent 200 is directed into the angled branch vessel 114 so that the proximal end face 208 is more proximate to the vessel juncture 115 than the distal end face 209. In an embodiment, a constriction of the angled branch vessel 114 is featured adjacent the vessel juncture 115 and/or at a deployment site of the stent 200. In one embodiment, the constriction is due to plaque 116 provided along the interior wall 120 of the angled branch vessel 114 and opposite the fork or bifurcation 118 of the vessel juncture 115. However, in another embodiment, at the deployment site of the stent 200, the constriction is based on a narrowing of the angled branch vessel 114 (e.g. congenital vascular malformation) and deployment of the stent 200 at the deployment site is used to widen the angled branch vessel 114 and eliminate this narrowing.

In an embodiment, the non-orthogonal angle 210 of the stent 200 is determined based on the non-orthogonal angle 110 of the vessel juncture 115. In one embodiment, the non-orthogonal angle 210 is equal to or within a threshold (e.g. 10 degrees) of the non-orthogonal angle 110 of the vessel juncture 115. In yet another embodiment, a plurality of stents 200 are formed with a respective non-orthogonal angle 210 (e.g. 30 degrees, 45 degrees, 60 degrees) and the stent 200 is selected whose non-orthogonal angle 210 is more proximate to the non-orthogonal angle 110 of the vessel juncture 115. Thus, in one example embodiment, where the non-orthogonal angle 110 is about 42 degrees, a stent 200 with a non-orthogonal angle 210 of about 45 degrees is used rather than other stents 200 with non-orthogonal angles 210 of 30 degrees or 60 degrees. In yet another embodiment, the non-orthogonal angle 110 of the vessel juncture 115 is measured using one or more imaging systems and the stent 200 is formed with the non-orthogonal angle 210 based on the measured non-orthogonal angle 110 of the vessel juncture 115.

As depicted in FIG. 2B, the stent 200 is moved along the main vessel 112 over the guidewire 202 in the direction 217 until the stent 200 reaches the deployment site (FIG. 2C). In an embodiment, the stent 200 reaches the deployment site when the proximal end face 208 of the stent 200 reaches the plaque 116 along an axis of the angled branch vessel 114 and/or extends beyond the plaque 116. In an embodiment, the stent 200 reaches the deployment site when the proximal end face 208 of the stent 200 reaches the reduced inner diameter of the wall 120 of the angled vessel 114 adjacent the vessel juncture 115. In another embodiment, when the stent 200 reaches the deployment site the proximal end face 208 does not extend beyond a juncture axis 216 (e.g. and into the main vessel 112) that is defined by an upper boundary of the main vessel 112 extending through the vessel juncture 115. This ensures that the stent 200 and the proximal end face 208 do not extend into the main vessel 112 and thus advantageously does not affect a fluid flow (e.g. blood flow 125) in the main vessel 112.

FIG. 2D is an image that illustrates an example of the balloon 104′ of the assembly 210 of FIG. 2A being inflated to deploy the stent 200′ along the wall 120 of the angled branch vessel 114, according to an embodiment. In some embodiments, after deploying the stent 200′, the plaque 116 no longer extends into the angled branch vessel 114 and the angled branch vessel 114 is opened up by the deployed stent 200′. In other embodiments, after deploying the stent 200′, the reduced inner diameter of the wall 120 of the angled branch vessel 114 adjacent the vessel juncture 115 is eliminated and/or substantially reduced and the angled branch vessel 114 is opened up by the deployed stent 200′. As depicted in FIG. 2D, the inflated balloon 104′ extends into the main vessel 112, however the inflated balloon 104′ does not remain in the main vessel 112 and thus does not affect the flow of fluid (e.g. blood flow 125) in the main vessel 112. As depicted in FIG. 2E, the balloon 104 is deflated and is subsequently removed from the angled branch vessel 114 along the guidewire 102 through the main vessel 112 in the direction 219. Also, as depicted in FIG. 2E, the proximal end face 208 of the deployed stent 200′ does not extend into the main vessel 112 based on the non-orthogonal angle 210. FIG. 2F further depicts the removal of the balloon 104 through the main vessel 112 along the guidewire 102 and FIG. 2G depicts that the deployed stent 200′ advantageously does not extend into the main vessel 112 and thus does not affect the flow of fluid (e.g. blood flow 125) both along the main vessel 112 and into the angled branch vessel 114. This is in stark contrast with the conventional stent (FIGS. 1D-1E) where the end 122 of the deployed stent 106 extended into the main vessel 112 and affected the fluid flow (e.g. blood flow 125) in the main vessel 112. Additionally, FIG. 2G depicts that the deployed stent 200′ advantageously opens up the angled branch vessel 114 and does not affect the flow of fluid through the angled branch vessel 114. This is also in stark contrast with the conventional method depicted in FIG. 1G, where the compressed end 107 of the deployed stent 106 affected the flow of fluid in the angled branch vessel 114.

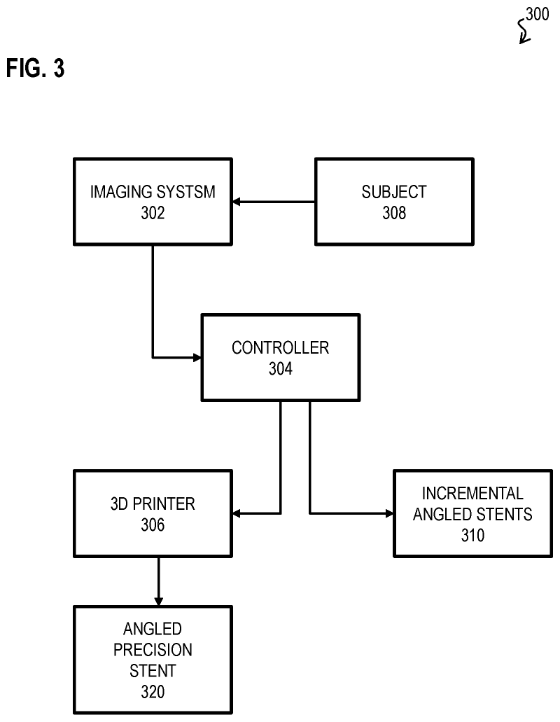

FIG. 3 is a block diagram that illustrates an example of a system 300 for forming the stent 200 of the assembly 210 of FIG. 2A, according to an embodiment. In an embodiment, the system 300 includes an imaging system 302 (e.g. MRI, CT scan, etc.) that is used to scan a subject 308 including generating an image of the vessel juncture 115 and/or measuring the angle 110 of the vessel juncture 115 in the subject 308 and/or measuring other dimensions of the vessel juncture 115 (e.g. inner diameter of the angled branch vessel 114, length of the plaque 116 along the interior wall 120, length of a reduced inner diameter of the wall 120 of the angled branch vessel 114 adjacent the juncture 115, etc) that may be used to form the stent 200. The subject 308 is not part of the system 300. In other embodiments, the imaging system 302 generates one or more of a coronary angiogram, a coronary CTA (Computed Tomography Angiography), MRA (Magnetic Resonance Angiography) and Intravascular Ultrasound (US). In an embodiment, the imaging system 302 is communicatively coupled with a controller 304 and transmits the image and/or data indicating the angle 110 and/or the other dimensions of the vessel juncture 115 to the controller 304. In an example embodiment, the image and/or data indicating the angle 110 and/or the other dimensions of the vessel juncture 115 are stored in a memory of the controller 304. In an embodiment, the controller 304 is a system that physicians use as a tool to determine the angle, diameter, and/or length of the stent. In another embodiment, the invention features a way to display the appropriate diagnostic image and/or utilize tools for collecting the measurement points. In an example embodiment, once the user has determined the points of measurement, the system will calculate and then produce the proper values for the angle, diameter, and/or length of the stent. The values will be delivered either to a 3D printer or uploaded to another system to produce the required equipment.

In an embodiment, the system 300 further includes a stent forming device, such as a 3D printer 306 that is configured to form stents 200. In an example embodiment, the 3D printer 306 is communicatively coupled with the controller 304 and the controller 304 transmits a signal to the 3D printer 306 with data indicating the angle 110 and/or the other dimensions of the vessel juncture 115. In an embodiment, the 3D printer 306 forms the stent 200 based on the data in the received signal from the controller 304. In an embodiment, the 3D printer 306 forms the stent 200 with the non-orthogonal angle 210 equal to or within a threshold angle (e.g. 5 degrees, 10 degrees, etc.) of the angle 110. As appreciated by one of ordinary skill in the art, the 3D printer 306 forms the stent 200 so that the diameter of the deployed stent 200′ is based on data in the controller 304 signal (e.g. the inner diameter of the angled branch vessel 114) and/or the length of the deployed stent 200′ is based on other data in the controller 304 signal (e.g. the length of the plaque 116 along the interior wall 120, the length of the reduced inner diameter of the wall 120 of the angled branch vessel 114 adjacent the juncture 115, etc.). In an example embodiment, the inner diameter of the angled branch vessel 114 is in a range from about 2 mm to about 20 mm, an outer diameter of the compressed stent 200 is in a range from about 1 mm to about 10 mm and an outer diameter of the deployed stent 200′ is in a range from about 2 mm to about 20 mm. In an example embodiment, other stents are sized to accommodate branch vessels one or more of trachea-bronchial, gastrointestinal and urological vessels. In an embodiment, the 3D printer 306 forms an angled precision stent 320, which is a stent 320 whose non-orthogonal angle 210 is sized or customized based on the subject 308 (e.g. based on the measured angle 110 of the vessel juncture 115 of the subject 308). Thus, the angled precision stent 320 is distinct from the incremental angled stent 310 discussed below, which are pre-cut at predetermined angles (e.g. incrementally spaced angles such as 30 degrees, 45, degrees, 60 degrees).

In an embodiment, the system 300 includes incremental angled stents 310 which each have the proximal end face 208 angled at an incremental or respective angle 210. In an example embodiment, the incremental angled stents 310 are made with a plurality of angles 210 (e.g. 30 degrees, 45 degrees, 60 degrees) that are incrementally spaced (e.g. 15 degrees). In this embodiment, the controller 304 transmits a signal that includes data indicating the angle 110 of the vessel juncture 115 and this angle 110 is used to select one of the incremental angled stents 310 for deployment in the angled branch vessel 114. In an embodiment, the incremental angled stent 310 is selected based on that stent 310 whose non-orthogonal angle 210 (e.g. 45 degrees) is most proximate to the angle 110 of the vessel juncture 115 (e.g. 40 degrees) from among all of the incremental angled stent 310 angles (e.g. 30 degrees, 45 degrees, 60 degrees). In some embodiments, the system 300 is used to form the incremental angled stents 310, such as by the controller 304 sending signals to the 3D printer 306 with data indicating one or more non-orthogonal angle values (e.g. 30 degrees, 45 degrees, 60 degrees) and the 3D printer 306 forms the incremental angled stents 310 with each non-orthogonal angle 210 value. In other embodiments, the pre-made stents 310 are formed by precision cutting a conventional stent (e.g. stent 108) to accommodate the angle 110 of the vessel juncture 115 without the 3D printer 306. In one example embodiment, to form the incremental angled stent 310 the conventional stent is cut at one end based on the angle 110, so that one end of the conventional stent forms the angle 210 with the longitudinal axis of the stent. Any cutting method appreciated by one of ordinary skill in the art can be used to cut the end of the conventional stent (e.g. stent 108) to form the angle 210 with the longitudinal axis of the stent.

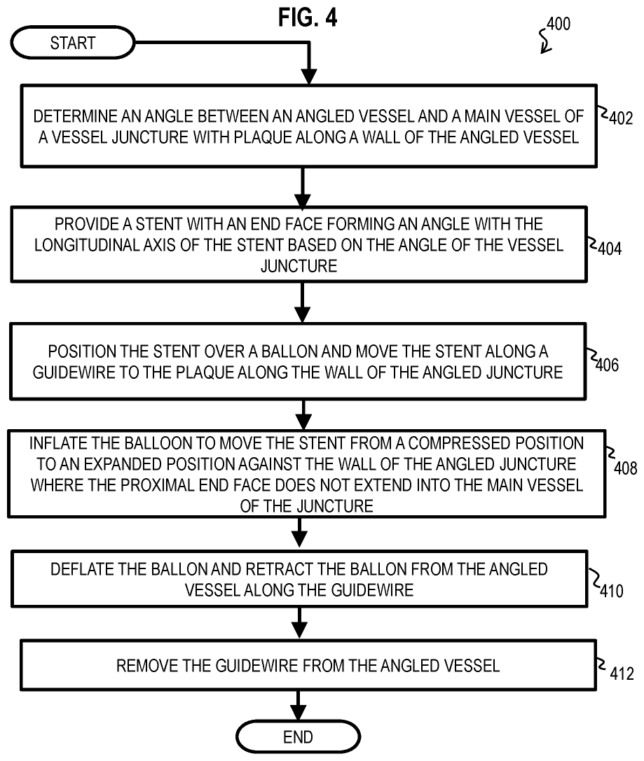

FIG. 4 is a flowchart illustrates an example of a method 400 for deploying a stent 200 along the main vessel and into an angled branch vessel 114 of a vessel juncture 115, according to an embodiment. Although steps are depicted in FIG. 4 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 402, the angle 110 between the angled branch vessel 114 and the main vessel 112 of the vessel juncture 115 is determined. In an embodiment, the imaging system 302 is used to determine the angle 110. In an example embodiment, the imaging system 302 automatically measures the angle 110 and outputs data indicating the angle 110. In another example embodiment, the imaging system 302 generates an image of the vessel juncture 115 and a medical practitioner manually measures the angle 110 based on the generated image. In some embodiments, the angled branch vessel 114 features a constriction at or adjacent a vicinity of the vessel juncture 115. In one embodiment, the construction is plaque 116 on the vessel wall 120 at or adjacent or in a vicinity of the vessel juncture 115. In other embodiments, the constriction is a narrowing diameter of the angled branch vessel 114 in a vicinity of the vessel juncture 115 and the system and method are employed to deploy the stent 200 to increase the narrowing diameter of the angled branch vessel 114 in the vicinity of the vessel juncture 115 so that the diameter of the angled branch vessel 114 does not narrow and/or is relatively constant in the vicinity of the vessel juncture 115.

In step 404, the stent 200 is provided with the proximal end face 208 that forms the non-orthogonal angle 210 with the longitudinal axis 202. In one embodiment, in step 404 the stent 200 is formed with the stent forming device (e.g. 3D printer 306). In an example embodiment, in step 404 the stent 200 is custom formed with the stent forming device (e.g. 3D printer 306 forms the angled precision stent 320) based on the measured angle 110 of the vessel juncture 115 by the imaging system 302. In another embodiment, in step 404 the stent 200 is selected among the incremental angled stents 310 based on which stent 310 has the closest non-orthogonal angle 210 to the non-orthogonal angle 110 of the vessel juncture 115. In an embodiment, in step 404 to form the incremental angled stent 310, the end face of a conventional stent 106, 108 is cut using any means appreciated by one of ordinary skill in the art so to form the non-orthogonal angle 210 between the proximal end face 208 and the longitudinal axis 202.

In step 406, the stent 200 is positioned over a deployment mechanism (e.g. balloon 104) and moved along the guidewire 102 through the main vessel 112 and into the angled branch vessel 114 to the deployment site (e.g. site of the constriction of the angled branch vessel 114). In one embodiment, the deployment site is at the constriction based on the plaque 116 along the interior wall 120 of the angled branch vessel 114 opposite to the fork or bifurcation 118 of the vessel juncture 115. In another embodiment, the deployment site is at the constriction caused by a narrowing of the angled branch vessel 114 adjacent to the vessel juncture 115. In an embodiment, the stent 200 is moved along the guidewire 102 until the proximal end face 208 is adjacent the plaque 116 (FIG. 2C) and/or until the proximal end face 208 reaches a proximal end of the plaque 116 (e.g. end of the plaque 116 that is most proximal to the juncture 115 or main vessel 112) along an axis of the angled branch vessel 114. In yet another embodiment, in step 406 the stent 200 is moved along the guidewire 102 until the stent 200 is adjacent the plaque 116 and does not extend beyond the juncture axis 216, i.e. does not extend into the main vessel 112. In another embodiment, the stent 200 is moved until the proximal end face 208 is adjacent the narrowing of the angled branch vessel 114.

In step 408, the balloon 104′ is inflated (FIG. 2D) to move the stent 200 from the compressed position to the expanded position against the wall 120 of the angled branch vessel 114. Additionally, in step 408, the stent 200′ is deployed so that the end face 208 does not extend into the main vessel 112 and/or so the end face 208 is aligned about parallel with the juncture axis 216 that defines an upper boundary of the main vessel 112 that is continuous through the vessel juncture 115. Although FIGS. 2C and 2E depict that the end face 208 is approximately aligned along the juncture axis 216, in other embodiments, the end face 208 is within the angled branch vessel 114 and about parallel to the juncture axis 216.

In step 410, the balloon 104 is deflated (FIGS. 2E-2F) and moved in the direction 219 along the guidewire 102 and out of the angled branch vessel 114 through the main vessel 112. In step 412, the guidewire 102 is removed from the angled branch vessel 114, leaving the deployed stent 200′ in the angled branch vessel 114.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article. As used herein, unless otherwise clear from the context, a value is “about” another value if it is within a factor of two (twice or half) of the other value. While example ranges are given, unless otherwise clear from the context, any contained ranges are also intended in various embodiments. Thus, a range from 0 to 10 includes the range 1 to 4 in some embodiments.

What is claimed is:

1. A method of deploying a stent along an angled branch vessel of a vessel juncture comprising:
a. determining a first non-orthogonal angle between the angled branch vessel and a main vessel of the vessel juncture, wherein the angled branch vessel features a constriction adjacent the vessel juncture;
b. providing a stent with an end face forming a second non-orthogonal angle with a longitudinal axis of the stent, said second non-orthogonal angle based on the first non-orthogonal angle of the vessel juncture;
c. moving the stent along a guidewire in the angled branch vessel to a deployment site based on the constriction of the angled branch vessel;
d. inflating a balloon positioned between the stent and the guidewire to move the stent from a compressed position to an expanded position against the wall of the angled branch vessel, wherein the end face does not extend into the main vessel of the vessel juncture;
e. deflating the balloon and retracting the balloon from the angled branch vessel along the guidewire; and
f. removing the guidewire from the angled vessel.

2. A method as recited in claim 1 wherein the constriction is based on plaque along a wall of the angled branch vessel adjacent the vessel juncture and wherein the plaque along the wall is opposite from a fork of the vessel juncture.

3. A method as recited in claim 1 wherein the constriction is based on a reduced inner diameter of a wall of the angled branch vessel adjacent the vessel juncture.

4. A method as recited in claim 1 wherein the first non-orthogonal angle and the second non-orthogonal angle are each between about 10 degrees and about 80 degrees.

5. A method as recited in claim 1, wherein the second non-orthogonal angle is within about 20 degrees of the first non-orthogonal angle.

6. A method as recited in claim 1, wherein the main vessel extends continuously through the vessel juncture and one end of the angled branch vessel terminates at the vessel juncture and wherein a diameter of the main vessel is greater than a diameter of the angled branch vessel.

7. A method as recited in claim 1, wherein the determining the angle comprises scanning a subject with an imaging system to produce an image including the vessel juncture and measuring the first non-orthogonal angle between the angled branch vessel and the main vessel of the vessel juncture in the image.

8. A method as recited in claim 1, wherein the providing the stent comprises providing a plurality of stents, wherein the end face of each stent forms a respective second non-orthogonal angle with the longitudinal axis and selecting a stent among the plurality of stents, wherein the second non-orthogonal angle of the selected stent is the most proximate to the first non-orthogonal angle among the plurality of stents.

9. A method as recited in claim 1, wherein the providing the stent comprises forming, with a 3D printer, the stent with the end face with the second-nonorthogonal angle based on the first non-orthogonal angle.

10. A stent for deployment along an angled branch vessel of a vessel juncture, said vessel juncture comprising the angled branch vessel and a main vessel with a first non-orthogonal angle between the angled branch vessel and the main vessel, said stent comprising:
a proximal end face;
a distal end face; and
a central portion between the proximal end face and the distal end face, said central portion defining a longitudinal axis;
wherein the proximal end face forms a second non-orthogonal angle relative to the longitudinal axis that is based on the first non-orthogonal angle.

11. A stent as recited in claim 10, wherein the first non-orthogonal angle and the second non-orthogonal angle are each between about 10 degrees and about 80 degrees.

12. A method as recited in claim 10, wherein the first non-orthogonal angle and the second non-orthogonal angle are each between about 20 degrees and about 70 degrees.

13. A stent as recited in claim 10, wherein the second non-orthogonal angle is within about 20 degrees of the first non-orthogonal angle.

14. A stent as recited in claim 12, wherein the central portion is cylindrical and wherein the distal end face forms an orthogonal angle relative to the longitudinal axis.

15. A stent as recited in claim 12, further comprising a first stent and a second stent, wherein the second non-orthogonal angle of the first stent is different than the second non-orthogonal angle of the second stent.

16. A stent as recited in claim 15, wherein the second non-orthogonal angle comprises at least one of about 30 degrees, about 45 degrees and about 60 degrees.

17. A stent as recited in claim 12, wherein the stent is formed from at least one of metal alloy, bio-absorbing and bio-degradable material.

18. A system for deploying a stent along an angled branch vessel of a vessel juncture, said vessel juncture comprising the angled branch vessel and a main vessel with a first non-orthogonal angle between the angled branch vessel and the main vessel, said system comprising:
the stent of claim 10;
a guidewire positioned in the angled branch vessel such that the stent is configured to be moved along the guidewire to a constriction of the angled branch vessel; and
a balloon positioned between the stent and the guidewire and is configured to inflate to move the stent from a compressed position to an expanded position against the wall of the angled branch vessel, and wherein the end face does not extend into the main vessel of the vessel juncture;
wherein the balloon is further configured to be deflated and retracted from the angled branch vessel along the guidewire and the guidewire is configured to be removed from the angled branch vessel.

19. A system as recited in claim 18, further comprising an imaging system configured to scan a subject and generate an image including the vessel juncture.

20. A system as recited in claim 18, further comprising a 3D printer to form the stent with the end face oriented at the second non-orthogonal angle relative to the longitudinal axis.
